(19) United States
(12) Patent Application Publication
(10) Pub. No.: US 2024/0117435 A1
(43) Pub. Date: Apr. 11, 2024

(54) SYSTEMS AND METHODS FOR PERFORMING METHYLATION-BASED RISK STRATIFICATION FOR MYELODYSPLASTIC SYNDROMES

(71) Applicant: GRAIL, LLC, Menlo Park, CA (US)

(72) Inventors: Qinwen LIU, Fremont, CA (US); Alvin SHI, Palo Alto, CA (US); Oliver Claude VENN, San Francisco, CA (US); Gordon CANN, Redwood City, CA (US)

(73) Assignee: GRAIL, LLC, Menlo Park, CA (US)

(21) Appl. No.: 18/481,322

(22) Filed: Oct. 5, 2023

Related U.S. Application Data
(60) Provisional application No. 63/413,539, filed on Oct. 5, 2022.
(60) Provisional application No. 63/505,641, filed on Jun. 1, 2023.
(60) Provisional application No. 63/516,320, filed on Jul. 28, 2023.

(51) Int. Cl. C12Q 1/6883; G16H 50/20

(52) U.S. Cl. CPC C12Q 1/6883; G16H 50/20; C12Q 2600/154

(57) ABSTRACT

Systems and methods for predicting survival outcomes in patients diagnosed with Myelodysplastic Syndrome (MDS) are disclosed. One method may include: receiving DNA sequencing data derived from a methylation assay performed on a biological sample associated with the at least one patient; computing methylation beta-values for one or more CpG-sites identified in the sequencing data; identifying one or more differentially methylated regions (DMRs) based on statistical analysis of the methylation beta-values for the one or more CpG-sites; selecting, via a feature selection process, a subset of the one or more DMRs to utilize as training data; and training, using the training data, the classifier to predict the survival outcome of the at least one patient. Other aspects are described and claimed.

### CROSS REFERENCE TO RELATED APPLICATIONS

This application claims the benefit of priority to U.S. Provisional Patent Application No. 63/413,539, filed Oct. 5, 2022; U.S. Provisional Patent Application No. 63/505,641, filed Jun. 1, 2023; and U.S. Provisional Patent Application No. 63/516,320, filed Jul. 28, 2023, each of which is hereby incorporated by reference herein in its entirety.

### TECHNICAL FIELD

The present disclosure relates generally to the field of diagnostics and, more specifically, to systems and methods for predicting survival outcomes in patients diagnosed with certain diseases using trained machine learning models.

### BACKGROUND

Myelodysplastic Syndromes (MDS) are a diverse group of hematological disorders that originate in the bone marrow and manifest as abnormal differentiation and maturation of blood cells, leading to increased risk of progression to Acute Myeloid Leukemia (AML). Accurate prediction of survival outcomes in MDS patients is crucial for determining appropriate treatment strategies, patient counseling, and personalized management. Although prognostic techniques currently exist, there is a need for more accurate, reliable, and less invasive methods for predicting survival outcomes in MDS patients.

The present disclosure is accordingly directed to systems and methods that may leverage machine learning and data analytics for predicting survival outcomes in patients diagnosed with MDS. The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for utilizing methylation-based analysis to perform risk stratification in patients diagnosed with MDS.

In one aspect, a computer-implemented method for building a classifier to predict a survival outcome in at least one patient diagnosed with Myelodysplastic Syndrome (MDS) is provided. The computer-implemented method may include: receiving, at a computing device, DNA sequencing data derived from a methylation assay performed on a biological sample associated with the at least one patient; computing, using a processor associated with the computing device, methylation beta-values for one or more CpG-sites identified in the sequencing data; identifying, using the processor, one or more differentially methylated regions (DMRs) based on statistical analysis of the methylation beta-values for the one or more CpG-sites; selecting, using the processor and via a feature selection process, a subset of the one or more DMRs to utilize as training data; and training, using the processor and the training data, the classifier to predict the survival outcome of the at least one patient.

In another aspect, a system for building a classifier to predict a survival outcome in at least one patient diagnosed with Myelodysplastic Syndrome (MDS) is provided. The system may include: one or more processors; one or more computer readable media storing instructions that are executable by the one or more processors to perform operations to: receive, at a computing device associated with the system, DNA sequencing data derived from a methylation assay performed on a biological sample associated with the at least one patient; compute, using the one or more processors, methylation beta-values for one or more CpG-sites identified in the sequencing data; identify, using the one or more processors, one or more differentially methylated regions (DMRs) based on statistical analysis of the methylation beta-values for the one or more CpG-sites; select, using the one or more processors and via a feature selection process, a subset of the one or more DMRs to utilize as training data; and train, using the one or more processors and the training data, the classifier to predict the survival outcome of the at least one patient.

In yet another aspect, a non-transitory computer-readable medium storing computer-executable instructions is provided. The non-transitory computer-readable medium stores computer-executable instructions which, when executed by a system, cause the system to perform operations that may include: receiving, at a computing device associated with the system, DNA sequencing data derived from a methylation assay performed on a biological sample associated with at least one patient; computing, using a processor associated with the computing device, methylation beta-values for one or more CpG-sites identified in the sequencing data; identifying, using the processor, one or more differentially methylated regions (DMRs) based on statistical analysis of the methylation beta-values for the one or more CpG-sites; selecting, using the processor and via a feature selection process, a subset of the one or more DMRs to utilize as training data; and training, using the processor and the training data, a classifier to predict a survival outcome of the at least one patient.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

The accompanying drawings, which are incorporated in and constitute a part of this specification, illustrate several embodiments and together with the description, serve to explain the principles of the disclosure.

FIG. 1 depicts an exemplary system environment for an MDS risk stratification system, according to one or more embodiments of the present disclosure.

FIGS. 2A, 2B, and 2C depict graphs showing PCA of WGBS beta values illustrating stratification by survival status, according to one or more embodiments of the present disclosure.

FIGS. 3A, 3B, and 3C depict graphs showing PCA of WGBS beta values illustrating stratification by AML vs MDS status, according to one or more embodiments of the present disclosure.

FIGS. 4A, 4B, and 4C depict graphs showing PCA of CpG beta values illustrating stratification by survival outcomes, according to one or more embodiments of the present disclosure.

FIGS. 5A, 5B, and 5C depict graphs showing PCA of CpG beta values illustrating stratification by AML vs MDS status, according to one or more embodiments of the present disclosure.

FIG. 6 depicts graphs illustrating DMR analysis data for bone marrow WGBS, according to one or more embodiments of the present disclosure.

FIG. 7 depicts graphs illustrating DMR analysis data for serum TM per CpG, according to one or more embodiments of the present disclosure.

FIGS. 8A and 8B depict graphs illustrating enriched WGBS bone marrow DMRs, according to one or more embodiments of the present disclosure.

FIGS. 9A and 9B depict graphs illustrating enriched serum TM DMRs, according to one or more embodiments of the present disclosure.

FIG. 10 depicts a DMR beta-value heatmap, according to one or more embodiments of the present disclosure.

FIG. 11 provides validation data for a WGBS beta-value PC Random Forest classifier, according to one or more embodiments of the present disclosure.

FIG. 12 provides validation data for a serum TM beta-value PC Random Forest classifier, according to one or more embodiments of the present disclosure.

FIGS. 13A and 13B depict graphs that present known aspects of MDS biology upon analysis of DMRs in blood serum TM and bone marrow WGBS, according to one or more embodiments of the present disclosure.

FIGS. 14A and 14B depict graphs that illustrate biological pathway enrichment via KEGG for both blood serum TM and bone marrow WGBS DMRs, according to one or more embodiments of the present disclosure.

FIGS. 15A and 15B depict graphs that provide methylation performance data as compared to IPSS-R data, according to one or more embodiments of the present disclosure.

FIG. 16 depicts a graph showing that serum TM methylation features can be leveraged to generate personalized survival predictions, according to one or more embodiments of the present disclosure.

FIG. 17 depicts an exemplary process flow for building a classifier to predict a survival outcome in at least one patient diagnosed with MDS, according to one or more embodiments of the present disclosure.

FIG. 18A depicts a graph presenting a relationship between the percentage of HMA−/HMA+ samples in each IPSS-R level, according to one or more embodiments of the present disclosure.

FIG. 18B depicts a graph presenting survival rates associated with both the HMA− and HMA+ groupings, according to one or more embodiments of the present disclosure.

FIG. 19 depicts a PCA plot illustrating methylation beta values in an uncorrected blood serum TM dataset, according to one or more embodiments of the present disclosure.

FIGS. 20A and 20B depict graphs that illustrate the effects of HMA correction in blood serum TM samples, according to one or more embodiments of the present disclosure.

FIGS. 21A and 21B depict graphs that illustrate that stratification by survival still remains after regressing out the HMA effect in serum TM samples, according to one or more embodiments of the present disclosure.

FIG. 22 depicts an uncorrected DMR beta-value heatmap based on a serum TM dataset, according to one or more embodiments of the present disclosure.

FIG. 23 depicts a corrected DMR beta-value heatmap based on the serum TM dataset, according to one or more embodiments of the present disclosure.

FIG. 24 depicts a graph that illustrates performance of a classifier using HMA-corrected M-value for serum TM, according to one or more embodiments of the present disclosure.

FIGS. 25A and 25B depict graphs that illustrate the effects of HMA correction in bone marrow WGBS samples, according to one or more embodiments of the present disclosure.

FIGS. 26A and 26B depict graphs that illustrate that stratification by survival still remains after regressing out the HMA effect in bone marrow WGBS samples, according to one or more embodiments of the present disclosure.

FIG. 27 depicts an uncorrected DMR beta-value heatmap based on a bone marrow WGBS dataset, according to one or more embodiments of the present disclosure.

FIG. 28 depicts a corrected DMR beta-value heatmap based on the bone marrow WGBS dataset, according to one or more embodiments of the present disclosure.

FIG. 29 depicts a graph that illustrates performance of a classifier using HMA-corrected M-value for bone marrow WGBS, according to one or more embodiments of the present disclosure.

FIG. 30 depicts an example computing system, according to one or more aspects of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

Risk stratification at diagnosis for myelodysplastic syndromes (MDS) is critical to informing clinical decision making with regards to treatment administration. The current gold-standard MDS risk stratification approach is the Revised International Prognostic Scoring System (IPSS-R). This system requires a bone marrow aspirate and involves the use of five variables (cytogenics, blast percent, absolute neutrophil counts, platelet level, and hemoglobin counts) to categorize patients into 1 of 5 groups, from very low risk to very high risk (e.g., based on risk of mortality and transformation to AML).

Although the conventional IPSS-R technique has been valuable in risk stratification and treatment decision-making, it is not without its issues and limitations. For example, IPSS-R categorizes patients into relatively broad risk groups based on clinical and laboratory parameters. This approach may lead to limited precision in predicting individual patient outcomes. Additionally, because IPSS-R relies on clinical and hematological parameters to determine risk categories, such as blood cell counts and bone marrow blast percentage, it has limited incorporation of molecular markers that can provide additional insights into disease progression. Furthermore, in a similar vein, because MDS is a heterogenous disease with diverse molecular and genetic alterations, IPSS-R does not fully capture this heterogeneity, which may lead to potential underestimation or overestimation of risk for certain patient subgroups. A need therefore exists for more accurate and reliable methods for predicting survival outcomes in MDS patients.

Accordingly, the present disclosure provides a novel approach for predicting survival outcomes in patients diagnosed with MDS. More particularly, the method may involve methylation-based analysis of genomic DNA to identify specific DNA methylation patterns associated with MDS progression and patient survival. Specifically, by incorporating methylation patterns using whole-genome bisulfite sequencing (WGBS) of bone marrow tissue and/or a targeted methylation (TM) assay on a liquid biopsy sample, such as a blood sample (e.g., serum, plasma, or a whole blood sample), the embodiments described herein may capture molecular heterogeneity and enhance the accuracy of survival outcome predictions. Although examples herein refer to blood serum in relation to initial testing data, the specification should not be limited as such, and it is contemplated that any portion of a blood sample, or a whole blood sample, may be used in the methods and systems described herein.

Furthermore, artificial intelligence (AI) and machine learning techniques may be employed to develop predictive models that may capture relationships between specific methylation patterns and survival outcomes. These models may thereafter be utilized to provide more precise and/or personalized MDS risk predictions. The system described herein may provide a less-invasive and less-painful approach to MDS risk stratification, as compared to a bone marrow aspirate. In some aspects, the accuracy of the methods described herein may be on par with or superior to those of the current IPSS-R approach, thereby improving the patient experience and potentially replacing or supplementing IPSS-R as an MDS risk stratification measure.

In view of the foregoing, the methylation-based profiling concepts described herein present a promising alternative for MDS risk stratification, as MDS-related somatic mutations may affect key epigenetic regulators (e.g., TET2, DNMT3A, IDH1, IDH2, and WT1), which may lead to downstream epigenetic changes.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

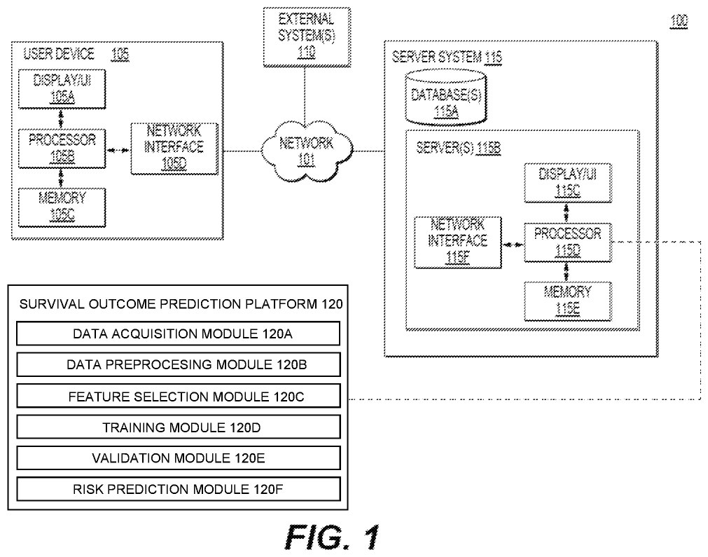

FIG. 1 depicts an exemplary system environment 100 that may be utilized with the MDS risk stratification techniques presented herein. One or more user device(s) 105, one or more external system(s) 110, and one or more server system(s) 115 may communicate across a network 101. As will be discussed in further detail below, one or more server system(s) 115 may communicate with one or more of the other components of the environment 100 across the network 101. The one or more user device(s) 105 may be associated with a user, e.g., a system manager, a research scientist, a healthcare provider (e.g., a doctor, etc.), and the like. Although depicted as separate components in FIG. 1, it should be understood that a component or portion of a component in the environment 100 may, in some embodiments, be integrated with or incorporated into one or more other components. For example, a portion of the display 115C may be integrated into the user device 105 or the like. In some embodiments, operations or aspects of one or more of the components listed above may be distributed amongst one or more other components. Any suitable arrangement and/or integration of the various systems and devices of the environment 100 may be used.

In some embodiments, the components of the environment 100 may be associated with a common entity (e.g., a single business or organization, etc.). Alternatively, one or more of the components may be associated with a different entity than another. The systems and devices of the environment 100 may communicate in any arrangement. For example, one or more user device(s) 105 may be associated with one or more clients or service subscribers, and server system(s) 115 may be associated with a service provider responsible for receiving raw datasets from the one or more clients or service subscribers. As will be discussed herein, systems and/or devices of the environment 100 may communicate in order to collect and aggregate data from various sources (e.g., databases containing patient sample data, online sources containing MDS-related data, user inputs, etc.), train one or more machine learning models based on the aggregated data, and leverage the trained models to generate survival outcome predictions for patients diagnosed with MDS.

The user device(s) 105 may be configured to enable the user to access and/or interact with other systems in the environment 100. For example, the user device(s) 105 may be a computer system such as, for example, a desktop computer, a mobile device, a tablet device, a wearable device, etc. The user device(s) 105 may include a display/user interface (UI) 105A, a processor 105B, a memory 105C, and/or a network interface 105D. The user device(s) 105 may execute, by the processor 105B, an operating system (O/S) and at least one electronic application (each stored in memory 105C). The electronic application may be a desktop program, a browser program, a web client, or a mobile application program (which may also be a browser program in a mobile O/S), system control software, system monitoring software, software development tools, or the like. In some embodiments, the electronic application(s) may be associated with one or more of the other components in the environment 100. The application may manage the memory 105C, such as a database, to transmit streaming data to network 101. The display/UI 105A may be a touch screen or a display with other input systems (e.g., mouse, keyboard, etc.) so that the user(s) may interact with the application and/or the O/S. The network interface 105D may be a TCP/IP network interface for, e.g., Ethernet or wireless communications with the network 101. The processor 105B, while executing the application, may generate data and/or receive user inputs from the display/UI 105A and/or receive/transmit messages to the server system 115, and may further perform one or more operations prior to providing an output to the network 101.

The electronic application, executed by the processor 105B of the user device 105, may generate one or many points of data that can be accessed, viewed, and/or interacted with by a user of the user device(s) 105. As an example, the electronic application may be an MDS risk stratification application that executes on the user device(s) 105 but may be managed by the server system 115. A user of the user device(s) 105 may interact with the MDS risk stratification application to modify parameters of a machine learning model, provide patient input, receive treatment recommendations based on a presented risk score and/or survival outcome prediction, and the like.

External systems 110 may be, for example, one or more third party and/or auxiliary systems that integrate and/or communicate with the server system 115 in performing various information extraction tasks. External systems 110 may be in communication with other device(s) or system(s) in the environment 100 over the one or more networks 101. For example, external systems 110 may communicate with the server system 115 via API (application programming interface) access over the one or more networks 101, and also communicate with the user device(s) 105 via web browser access over the one or more networks 101. Non-limiting examples of possible external systems 110 may include one or more data repositories containing patient information, DNA sequencing data, historical MDS survival outcome information, and the like.

In various embodiments, the network 101 may be a wide area network (“WAN”), a local area network (“LAN”), a personal area network (“PAN”), or the like. In some embodiments, network 101 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 115 may include an electronic data system, computer-readable memory such as a hard drive, flash drive, disk, etc. In some embodiments, the server system 115 includes and/or interacts with an application programming interface for exchanging data to other systems, e.g., one or more of the other components of the environment. The server system 115 may include and/or act as the host for an application platform (e.g., an MDS risk stratification application platform, etc.) that may be accessible by the user device(s) 105.

The server system 115 may include at least one database 115A and at least one server 115B. The server system 115 may be a computer, system of computers (e.g., rack server(s)), and/or or a cloud service computer system. The server system may store or have access to database 115A (e.g., hosted on a third party server or in memory 115E). The server(s) may include a display/UI 115C, a processor 115D, a memory 115E, and/or a network interface 115F. The display/UI 115C may be a touch screen or a display with other input systems (e.g., mouse, keyboard, etc.) for an operator of the server 115B to control the functions of the server 115B. The server system 115 may execute, by the processor 115D, an operating system (O/S) and at least one instance of a servlet program (each stored in memory 115E). When user device(s) 105 transmit input to the server system, the received dataset and/or dataset information may be stored in memory 115E or database 115A. The network interface 115F may be a TCP/IP network interface for, e.g., Ethernet or wireless communications with the network 101.

The processor 115D may include and/or execute instructions to implement a survival outcome prediction platform 120, which may include a data acquisition module 120A, a data preprocessing module 120B, a feature selection module 120C, a training module 120D, a validation module 120E, and a risk prediction module 120F. In an embodiment, the data acquisition module 120A, the data preprocessing module 120B, the feature selection module 120C, the training module 120D, the validation module 120E, and the risk prediction module 120F may all be contained within the server system 115, e.g., by the survival outcome prediction platform 120. Alternatively, some or all of the foregoing modules may be submodules of other modules within each other or may be resident on other components of the environment 100. For example, the data acquisition module 120A may be incorporated into an application resident on the user device 105 whereas the data preprocessing module 120B, the feature selection module 120C, the training module 120D, the validation module 120E, and the risk prediction module 120F may be contained within the survival outcome prediction platform 120.

The data acquisition module 120A may include instructions for collecting and aggregating data derived from whole-genome bisulfite sequencing (WGBS) of bone marrow tissue and/or from cell-free targeted methylation assay on collected sample types, e.g., a blood sample (e.g., a serum sample, a plasma sample, a whole blood sample), a urine sample, a saliva sample, a tissue sample, a bone marrow sample, etc. The methylation data obtained from both the WGBS approach and the cell-free TM assay may provide information on the DNA methylation patterns in the bone marrow tissue and blood samples, respectively. This data serves as the basis for subsequent analysis using machine learning models to predict survival outcomes in MDS patients. The data may be acquired by the actual performance of analytical techniques and/or may be acquired via data acquisition from other sources.

Using the WGBS approach, a high-throughput sequencing technique is provided that enables the assessment of DNA methylation at single-base resolution across the entire genome. Bone marrow tissue samples may be obtained from patients diagnosed with MDS (e.g., by performance of a bone marrow biopsy or aspiration procedure, etc.), and WGBS may be performed on the acquired samples. In WGBS, the DNA from the collected bone marrow tissue samples is treated with sodium bisulfite, which converts unmethylated cytosine residues to uracil while leaving methylated cytosines unchanged. This treatment distinguishes between methylated and unmethylated cytosines, allowing for the identification of specific methylation patterns. The bisulfite-treated DNA is then subjected to next-generation sequencing, where DNA fragments are sequenced, and the resulting sequences are aligned to a reference genome. The alignment data is utilized to determine the methylation status of individual CpG sites throughout the genome. Thereafter, methylation beta-values, which represent the degree of DNA methylation at a specific CpG site or region (and are calculated as the ratio of the methylated signal intensity to the sum of the methylated and umethylated signal intensities at each CpG site), may be computed. Beta values may range between 0 and 1, where 0 indicates no methylation, and 1 represents complete methylation. In an embodiment, the entire dataset may be leveraged by taking, e.g., 2,812,497×120 1 kb beta values matrix as input.

In an aspect, sample data was obtained from a cohort of 127 patients, from which 104 were afflicted with MDS and 23 were afflicted with AML. Using the cell-free TM assay approach, liquid samples, such as blood (e.g., plasma, serum, or whole blood) samples, collected from the patients may undergo a TM assay to assess DNA methylation patterns. Such an assay may focus on specific CpG sites of interest (e.g., those known to be associated with MDS) rather than providing a comprehensive view of the entire genome, as described above with respect to WGBS. Once the CpG sites of interest are identified, primers are designed to specifically amplify the regions containing these CpG sites. The cell-free DNA extracted from the blood samples may thereafter undergo bisulfite conversion, by which unmethylated cytosines are converted to uracil (e.g., via bisulfite treatment) while leaving methylated cytosines unchanged. The bisulfite-converted DNA is subjected to PCR amplification using the primers designed for the targeted CpG sites. PCR amplification specifically amplifies the regions of interest, including the CpG sites, from the bisulfite-converted DNA and ultimately enriches the targeted DNA regions for subsequent analysis. The PCR-amplified DNA fragments may then be subjected to methylation-specific analysis to determine the methylation status at the targeted CpG sites. Various techniques may be employed to facilitate this determination, e.g., targeted next-generation sequencing, which utilizes next-generation sequencing platforms to analyze the methylation patterns of the targeted CpG sites to determine methylation levels at the specific CpG sites of interest. Similar to the WGBS approach, the cell-free TM assay may generate information that may be presented as methylation beta-values, which provide a quantitative measure of methylation levels as the specific CpG sites.

The computed beta values may be used in differentially methylated region (DMR) analysis to compare methylation levels between groups and determine statistically significant differences. More particularly, DMRs are genomic regions that exhibit differential methylation patterns between different groups or conditions. These regions are identified based on the differential methylation patterns observed across multiple CpG sites within a genomic region and are defined based on statistical comparisons of beta values between different groups or conditions. Accordingly, DMR analysis involves comparing beta values between groups to identify regions with differential methylation. Various tests, such as t-tests, nonparametric tests, or linear regression models, can be used to assess the significance of methylation differences at individual CpG sites or regions. DMRs may be defined based on statistical thresholds, such as p-values or adjusted p-values, indicating significant differences in methylation levels between groups.

The data collected by the data acquisition module 120A may be passed to the data preprocessing module 120B to ensure compatibility and quality for model training. Stated differently, the data preprocessing module 120B may transform the sequencing data into a consistent and suitable format for training one or more machine learning models. The various steps involved in data preprocessing may include data cleaning (e.g., removal of any duplicate, incomplete, or erroneous entries from the dataset), missing value handling (e.g., resolving missing data points by employing appropriate techniques to estimate or fill in the missing values), normalization or standardization (e.g., rescaling the data to bring it to a common scale or distribution, which enables fair comparisons and prevents certain features from dominating the analysis due to their scales), and feature encoding (e.g., converting categorical variables into a numerical or binary representation that is suitable for machine learning models). It is important to note that the data preprocessing steps listed above may vary based upon the type of data collected by the data acquisition module 120A and/or by the type of machine learning model that will be trained on the preprocessed data. More particularly, the steps carried out by the data preprocessing module 120B may include more or less steps than those listed and described above.

The preprocessed data may be passed to the feature selection module 120C to identify and select the most relevant features from the cumulative dataset for model training. The feature selection process may reduce the dimensionality of the dataset by eliminating irrelevant or redundant features, which ultimately may improve model performance, facilitate faster model training and inference (i.e., working with a reduced set of features may reduce the computational complexity of training and inference processes), and contribute to enhanced model interpretation. In the context of the methylation approaches described herein, feature selection may involve the identification of a subset of DMRs, alongside individual CpG site beta values, that may be more relevant to the prediction of an individual's survival status. By incorporating both DMRs and beta values as features in the training and prediction process, the model may leverage the information from different scales of methylation data. DMRs capture larger-scale methylation patterns associated with specific genomic regions, whereas beta values provide detailed information about methylation levels at individual CpG sites. This combined approach allows for a comprehensive analysis of the methylation data and may improve the model's ability to capture the complexity and heterogeneity of methylation patterns associated with different outcomes.

In an embodiment, one approach that may be leveraged to perform feature selection may be principal component analysis (PCA), which is a statistical technique that may be applied to the preprocessed data to capture the most informative features. More particularly, high-dimensional datasets, such as those generated from methylation assays, may contain a large number of features (e.g., methylation beta-values) that can be computationally demanding and may suffer from issues like overfitting, which occurs when a model performs well on the training data but fails to generalize to new data. PCA may transform the original dataset into a new set of uncorrelated variables called principal components (PCs), which are linear combinations of the original features. By capturing the maximum variance in the data, PCA may allow for dimensionality reduction while retaining the most relevant information. After computing the PCs, PCA may allow for dimensionality reduction by selecting a subset of the components that capture the most relevant information, which may be achieved by retaining the top PCs that explain a significant portion of the total variance in the data. The reduced feature set obtained from PCA may replace the original high-dimensional feature set in subsequent steps, such as in building a random forest classifier, as further described below.

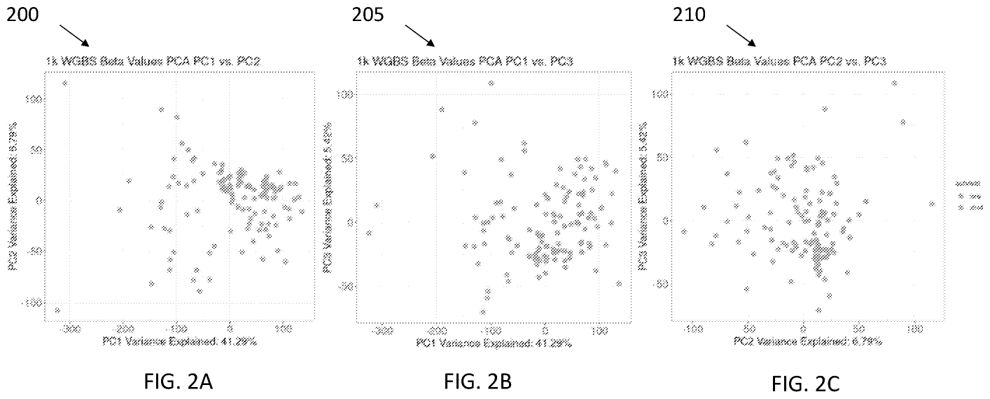

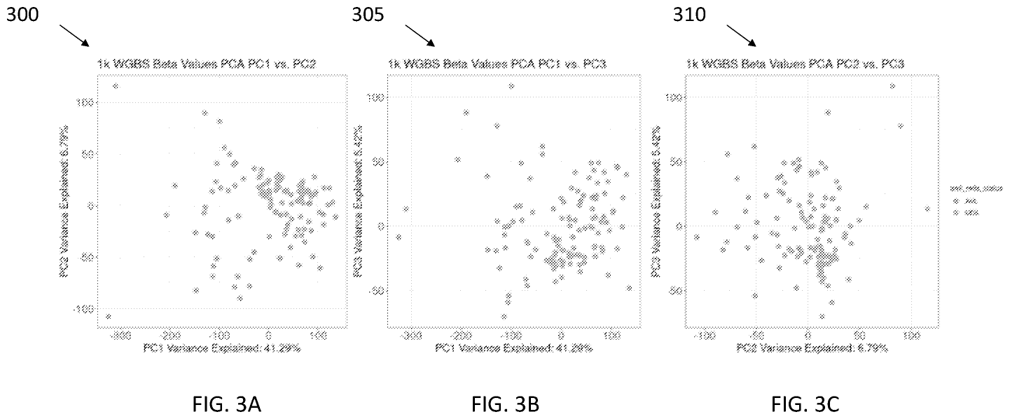

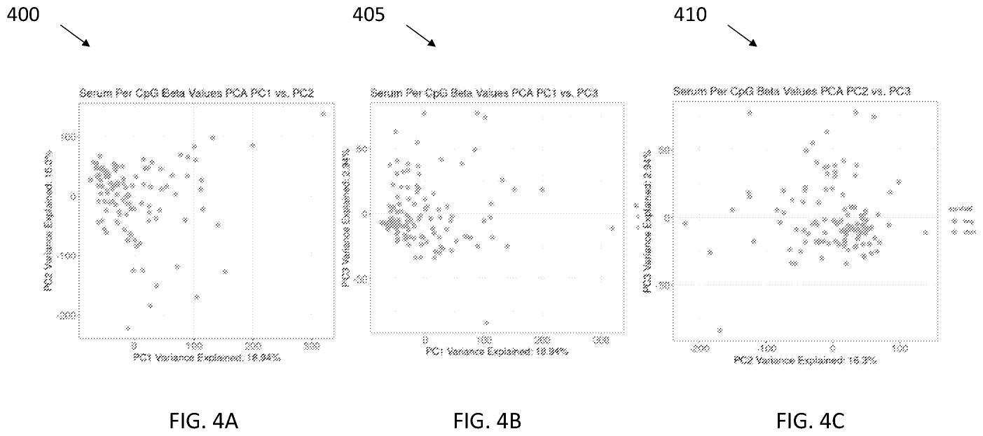

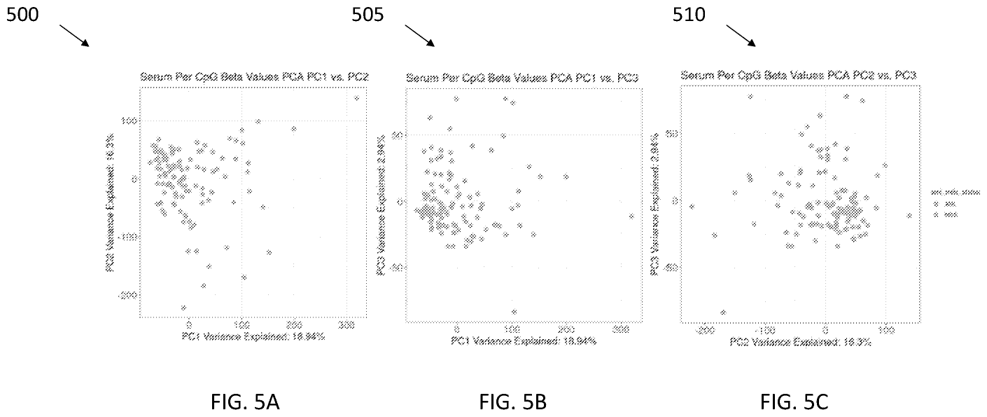

As a depiction of the foregoing, FIGS. 2A, 2B, and 2C depict graphs 200, 205, and 210 showing PCA of bone marrow WGBS beta values illustrating stratification by survival status. Short survival status was defined as a survival status of less than 3 years, and long survival status was defined as a survival status of more than 3 years. FIGS. 3A, 3B, and 3C depict graphs 300, 305, and 310 showing PCA of bone marrow WGBS beta values illustrating stratification by AML vs MDS status. FIGS. 4A, 4B, and 4C depict graphs 400, 405, and 410 showing PCA of blood serum CpG beta values illustrating stratification by survival outcomes. Again, short survival status was defined as a survival status of less than 3 years, and long survival status was defined as a survival status of more than 3 years. FIGS. 5A, 5B, and 5C depict graphs 500, 505, and 510 showing PCA of blood serum CpG beta values illustrating stratification by AML vs MDS status.

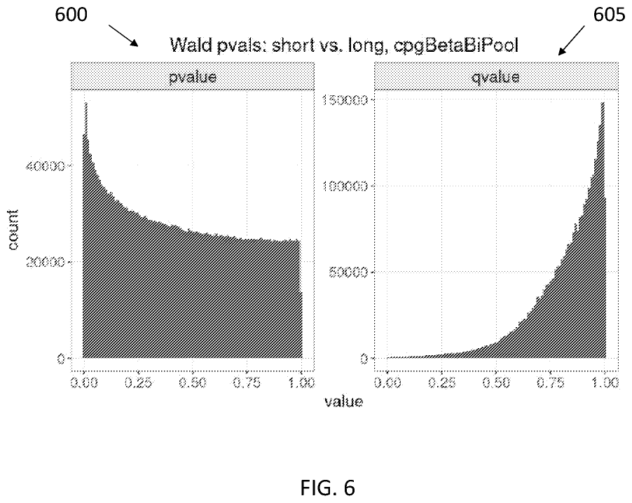

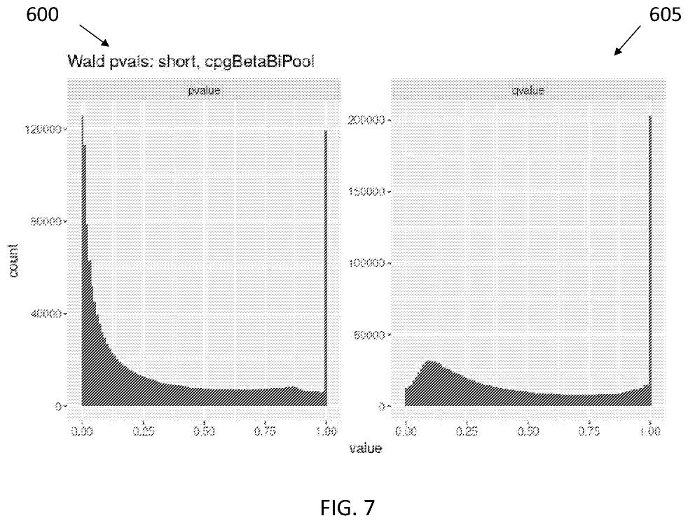

FIG. 6 illustrates graphs 600, 605 for DMR analysis that identify methylation biomarkers that differentiate survival for bone marrow WGBS. In the graphs of FIG. 6, 2,812,497 total 1 kb regions were analyzed. Aggregated CpG counts were treated as a beta-binomial. In total, 4,568 DMRs were identified, which equals approximately 0.1624% of the total regions analyzed (q-value <0.05). The analysis of graphs 600, 605 in FIG. 6 demonstrates that classification techniques described herein may enable DMR analysis at 1 kb intervals. FIG. 7 illustrates graphs 700, 705 for DMR analysis that identify methylation biomarkers that differentiate survival for serum TM. DMRs were analyzed at the per-CpG level using an exemplary classifier. Out of 1,662,938 CpGs analyzed, 83,351 (5.01%) were identified as being significant. For both FIGS. 6 and 7, short survival status was defined as a survival status of less than 3 years, and long survival status was defined as a survival status of more than 3 years.

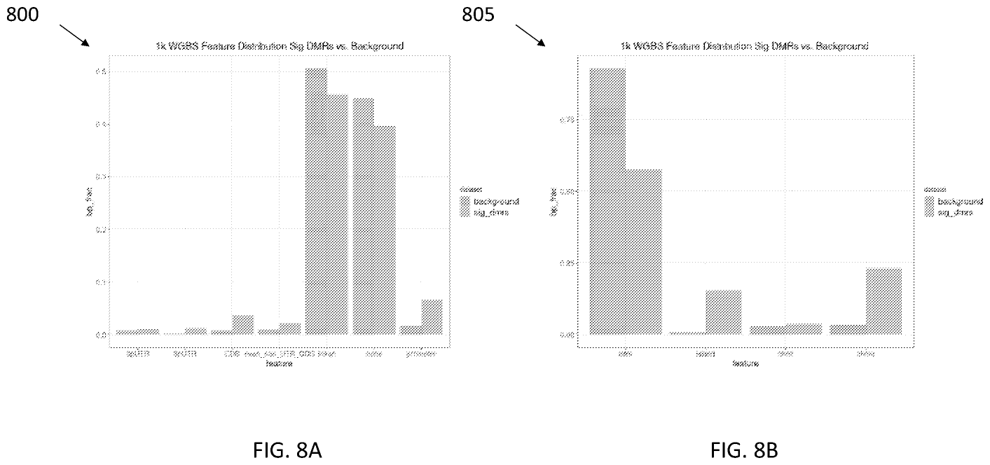

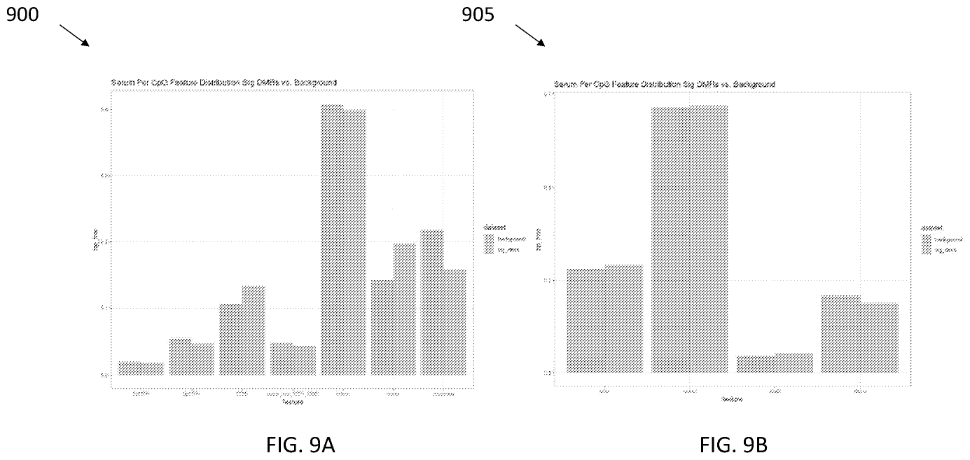

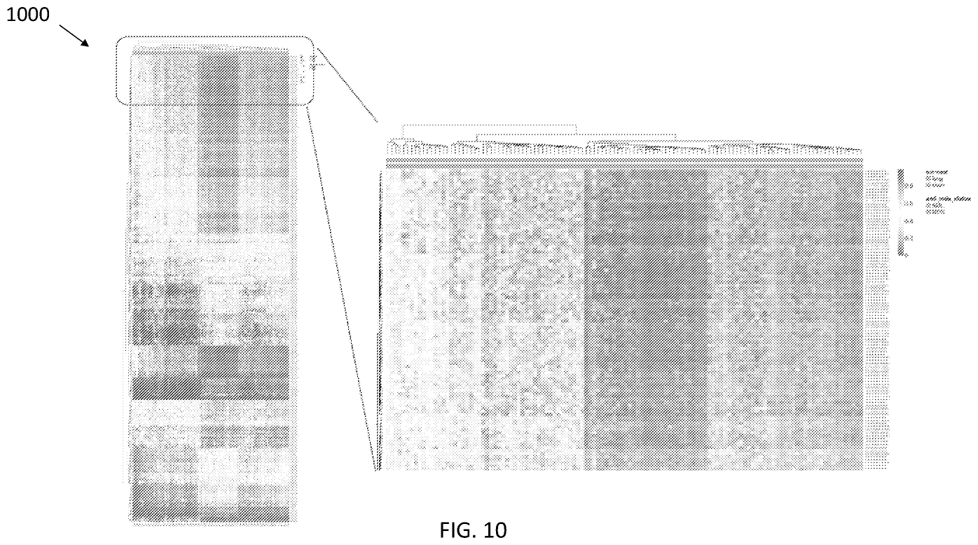

FIGS. 8A, 8B, 9A, and 9B provide additional information indicating that the methylation-based approaches described herein identify genomic regions that may be biologically meaningful through the identification of significant enriched biological pathways that are functionally important for tumor progression. Specifically, graphs 800 and 805 in FIGS. 8A and 8B, respectively, graphically depict WGBS feature distribution of significant DMRs vs. background. The data indicates that DMRs are enriched for CpG promoters, island, and shore regions. Graphs 900 and 905 in FIGS. 9A and 9B, respectively graphically depict blood serum per CpG feature distribution significant DMRs vs. background. The data indicates that blood serum DMR feature distribution shows no significant differences between significant DMRs and background. FIG. 10 illustrates a DMR beta-value heatmap 1000 showing clustering into three methylation blocks.

The lower dimensionality data containing the selected features may be utilized as training data to train the machine learning models via the training module 120D. In general, the training module 120D may train one or more machine learning models to recognize patterns and correlations between DMRs in bone marrow tissue and/or serum and corresponding survival outcomes in patients. In an embodiment, the training module 120D may include one or more machine learning models and/or instructions associated with each of the one or more machine learning models, e.g., instructions for generating, training, and/or using the machine learning models. The server system 115 may include instructions for retrieving output features, e.g., based on the input of the machine learning models, and/or operating the displays 105A and/or 115C to generate one or more output features. In some embodiments, a system or device other than the server system 115 may be used to generate and/or train the machine learning models. For example, such a system may include instructions for generating the machine learning model, the training data and/or ground truth, and/or instructions for training the machine learning model. A resulting trained machine learning model may then be provided to the server system 115.

In some embodiments, the machine learning models may be constructed using supervised learning, e.g., where a ground truth is known for the training data provided. The training may proceed by feeding a sample of training data into a model with variables set at initialized values. The model learns to capture the relationships between the input features and the corresponding target variable. For example, suppose feature extraction resulted in the obtainment of 10 principal components from the methylation data of MDS patients. These principal components correspond to the most significant variations in the methylation patterns associated with survival outcomes. To train a binary classification model, the survival outcomes need to be transformed into a binary label. To facilitate this, a threshold, or cutoff time, may be established to define the survival outcome as positive (e.g., the patient survived beyond the threshold) or negative (e.g., the patient did not survive beyond the threshold). For example, patients who survived beyond a threshold of three years may be labeled as “1” (positive outcome) while patients who did not survive beyond three years may be labeled as “0” (negative outcome). The training dataset is then prepared by combining the selected features (e.g., principal components) obtained from the methylation data with the corresponding binary survival outcome labels. Specifically, in this example, each patient's data entry includes the values of the 10 principal components labeled with their binarized threshold three-year survival outcome value. In an embodiment, the available dataset may be divided into two subsets: a training set and a testing set. The training set may be used to train the model, whereas the testing set may be used for model evaluation, as further described herein.

Although a variety of different types of machine learning architectures may be employed (e.g., convolutional neural networks, support-vector machines, gradient boosting machines, etc.), as a non-limiting designation, the type of machine learning architecture referenced throughout the remainder of this disclosure is a random forest classifier. A random forest is an ensemble learning method that combines multiple decision trees to make predictions. Each decision tree in the random forest is trained on a subset of the training data and a subset of the features. At each split within each tree, a subset of the CpG site beta values and/or DMR features are randomly selected for consideration. The random forest algorithm aggregates the predictions of all the individual trees to make the final prediction. The random forest classifier may therefore utilize the training dataset with the labeled survival outcomes to learn the relationship between the selected features and the survival outcome. Through this training, the random forest classifier may be trained to predict whether a patient is likely to survive beyond a defined time threshold (e.g., three years, etc.) based on the methylation patterns captured by the selected features. Once the random forest classifier is trained, it can be used to make predictions on new, unseen samples.

After the random forest classifier is constructed, the validation module 120E may assess its performance. More particularly, as an initial matter, the random forest classifier may make predictions on the testing set. The predicted outcomes on the testing set may be compared to the known outcomes (ground truth) to evaluate the performance of the classifier. More particularly, the classifier may be evaluated using appropriate metrics, such as area under the receiver operating characteristic curve (AUC-ROC), to assess the model's predictive capabilities. The AUC-ROC is a metric for classification accuracy of a binary predictive model across all score cutoffs. It measures a curve for all values of apparent true positive rates for equivalent false positive rates. It may vary from 0.5, indicating predictions are effectively random and the model has no predictive value, up to 1, indicating a perfectly predictive classifier.

To obtain a more robust assessment of the model's performance, a cross-validation evaluation technique may be employed to estimate the performance of the classifier on unseen data. Such a process may first involve dividing the available dataset into X equal-sized subsets, or folds, generally known as “K-folds.” Each fold contains a roughly equal distribution of samples across the different classes or outcomes. The cross-validation process involves “K” iterations, where each iteration uses K−1 folds for training and the remaining fold for testing. More particularly, one of the folds for each iteration is treated as the testing set, while the other K−1 folds are combined to form the training set. In each iteration, the model is trained on the training set using the chosen algorithm and hyperparameters. The trained model is then used to predict the outcomes of the samples in the testing fold. The predicted outcomes are compared to the known outcomes (ground truth) to evaluate the model's performance. The performance metrics obtained from each iteration (e.g., accuracy, precision, recall, etc.) are collected and the aggregated results provide an estimate of the model's performance across multiple test sets. These results help assess how well the model generalizes to unseen data and may provide a more reliable evaluation compared to a single train-test split, as described above.

In an embodiment, cross-validation, e.g., nested cross-validation, may also be used for hyperparameter tuning. Different combinations of hyperparameters may be evaluated using cross-validation, and the set of hyperparameters that yield the best performance may be selected. For example, the validation module 120E may be configured to iterate over different hyperparameter settings (e.g., maximum depth, number of trees) and evaluate the model's performance using cross-validation. The hyperparameter configuration that yields the best average performance (e.g., the highest AUC-ROC score, etc.) across the iterations may be selected as the “champion” or “optimal” hyperparameter configuration set.

TABLE 1

| AUROC | 5 PCs | 10 PCs | 20 PCs |
|---|---|---|---|
| 50 Trees | 0.7844 | 0.8141 | 0.8158 |
| 100 Trees | 0.7777 | 0.809 | 0.8098 |
| 200 Trees | 0.7817 | 0.8148 | 0.8165 |

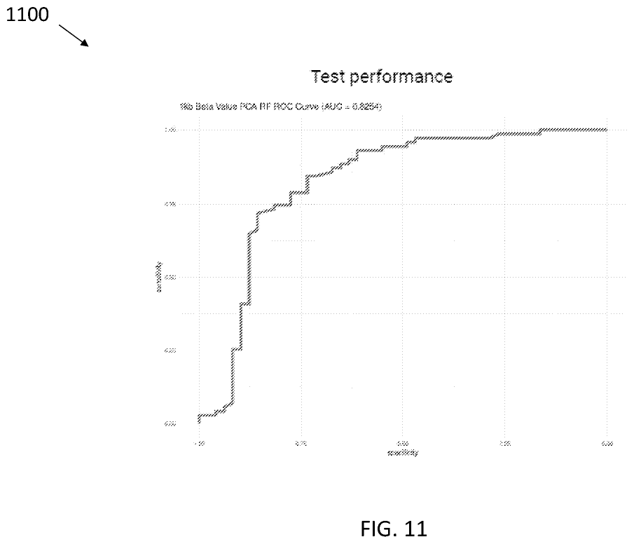

As an example of the foregoing, referring collectively to Table 1 above and graph 1100 in FIG. 11, hyperparameter optimization data is presented for the bone marrow WGBS validation set. As seen, the optimal hyperparameter configuration (i.e., the one configuration generating the highest AUROC score) is that which includes 20 principal components (e.g., as determined by the PCA) and 200 trees in the random forest classifier. This generated an AUROC score of 0.8165. Table 1 and graph 1100 in FIG. 11 indicate that WGBS beta-value PC random forest is predictive of survival status.

TABLE 2

| AUROC | 5 PCs | 10 PCs | 20 PCs |
|---|---|---|---|
| 50 Trees | 0.8028 | 0.7933 | 0.8257 |
| 100 Trees | 0.7822 | 0.7924 | 0.8192 |
| 200 Trees | 0.7988 | 0.7947 | 0.8144 |

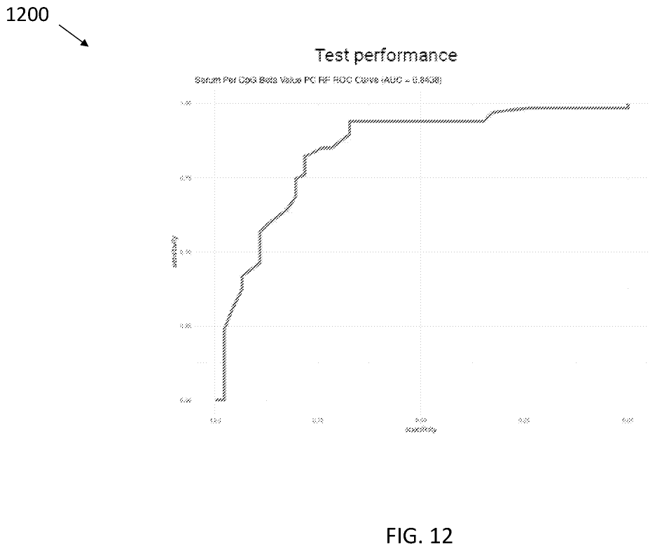

Similarly to the foregoing, referring collectively to Table 2 above and graph 1200 in FIG. 12, hyperparameter optimization data is presented for the blood serum TM validation set. As seen, the optimal hyperparameter configuration for the blood serum TM validation set is that which includes 20 principal components and 50 trees. This generated an AUROC score of 0.8257. Table 2 and graph 1200 in FIG. 12 indicate that serum TM beta values are predictive of survival status.

The risk prediction module 120F may leverage the trained and validated classifier to predict an individual's survival likelihood against a binarized (e.g., three-year) threshold. More particularly, the random forest classifier may be configured to generate a methylation prognostic score (MPS), which may represent a binary indication of short survival (e.g., under 3 years) or long survival (greater than 3 years) for individual's diagnosed with MDS. In an embodiment, these risk scores may further be used as inputs downstream to inform treatment plans.

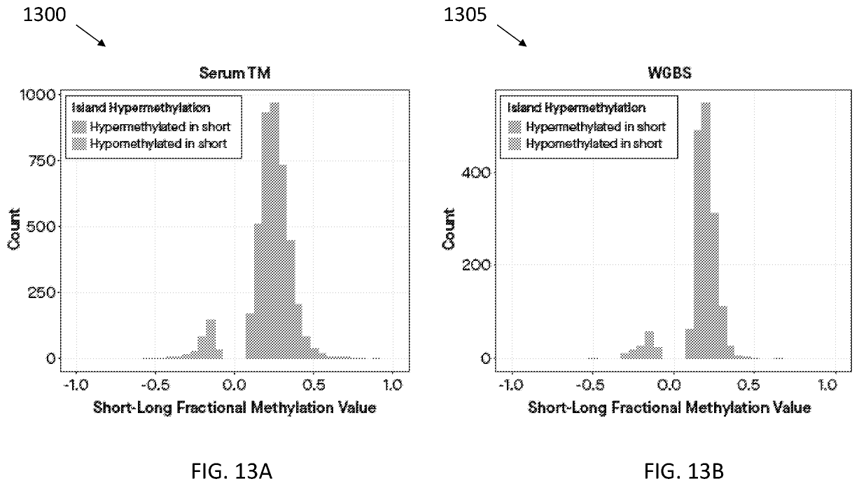

Referring now to FIGS. 13A and 13B, graphs 1300 and 1305 are depicted that reveal known aspects of MDS biology upon analysis of DMRs in blood serum TM and bone marrow WGBS. Specifically, graphs 1300A and 1300B illustrate the CpG Island hypermethylation signature in blood serum TM and bone marrow WGBS data, respectively. More particularly, significantly different DMRs between long and short survivor groups were identified in both blood serum TM (14,093 out of 103,142 (13.66%)) and bone marrow WGBS 7,742 out of 2,812,497 (0.27%)). As can be seen in graphs 1305 and 1310, CpG Island hypermethylation was more prevalent in the short survivor group compared to the long survivor group across both blood serum TM and bone marrow WGBS. These results indicate that CpG island hypermethylation is associated with AML progression.

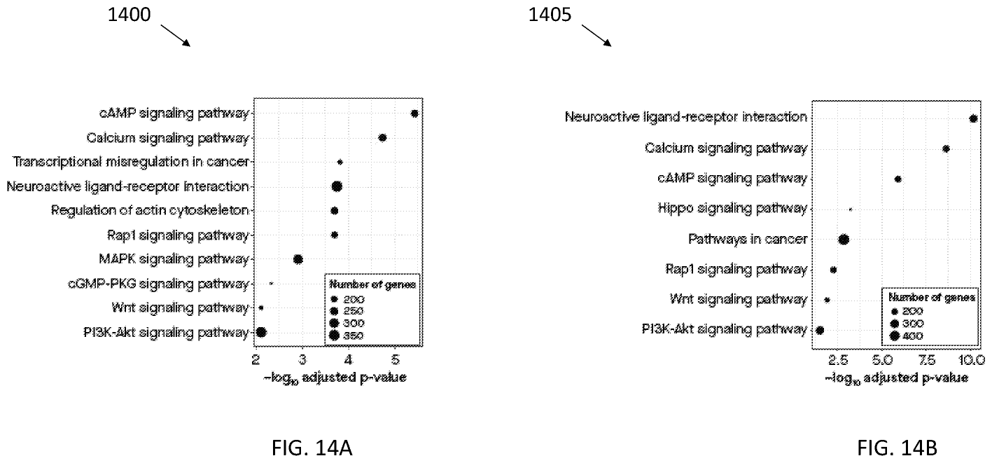

Referring now to FIGS. 14A and 14B, graphs 1400 and 1405 are presented that illustrate biological pathway enrichment via KEGG of serum TM DMRs and bone marrow WGBS DMRs, respectively. In both blood serum TM and bone marrow WGBS data, significant enrichment was observed for several pathways, such as cyclic adenosine 3,5,-monophosphate (cAMP), calcium, Ras-proximate-1 (Rapt), and Wnt signaling, all of which were previously implicated in MDS progression.

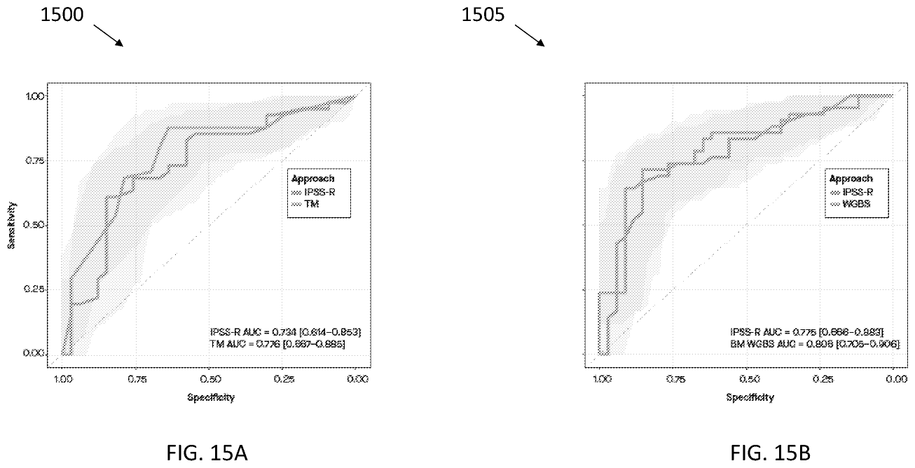

In an aspect, the ability of methylation-based features to predict binary short term vs long term overall survival using a methylation prognostic classifier was compared to the benchmark IPSS-R measure in a subset of 96 blood serum samples and 98 bone marrow samples from patients afflicted with MDS. Both bone marrow WGBS and blood serum TM had higher area-under-the-curve (AUC) values than IPSS-R alone for predicting binarized short term vs long term overall survival. More particularly, referring now to FIGS. 15A and 15B, graphs 1500 and 1505 are presented for blood serum TM and bone marrow WGBS, respectively. The graphs show that methylation feature analysis offer similar levels of risk prediction performance as the conventional IPSS-R approach. Specifically, both bone marrow WGBS and blood serum TM had higher AUC values than IPSS-R alone for predicting binarized under 3 year and over 3 year survival.

TABLE 3

| | |
|---|---|
| Serum TM | BM WGBS |

| | HR | p-value | HR | p-value |
|---|---|---|---|---|
| Methylation Prognostic | 4.86 | 0.002 | 4.82 | 0.016 |
| Score | | | | |
| IPSS Score | 1.30 | 0.003 | 1.29 | 0.003 |
| Age | 1.04 | 0.007 | 1.03 | 0.022 |
| Sex | 1.57 | 0.143 | 1.26 | 0.439 |

Referring now to Table 3 above, a multi-variable cox regression indicated that the MPS is a significant predictor of survival even after accounting for IPSS-R, age, and sex. Multivariable Cox regression may be utilized in the context of methylation data and survival analysis to assess the association between methylation-based features (such as DMRs or beta values) and survival outcomes while adjusting for other relevant variables (e.g., age, sex, etc.). More particularly, in many cases, survival outcomes may be influenced by various factors, such as age and sex, and multivariable Cox analysis allows for the control of these confounding factors, which indicates that the association between methylation features and survival outcomes is not distorted by the influence of other variables. This helps in obtaining more accurate and reliable estimates of the true effect of methylation on survival. For prognostics, by identifying the methylation features that significantly affect survival outcomes, a better understanding may be obtained on their impact on disease progression, treatment response, and overall patient prognosis.

TABLE 4

| | | | | | Overall |
|---|---|---|---|---|---|
| | IPSS-R | IPSS-R | | Age | Survival |
| | Category | Score | Gender | (years) | (years) |
| Patient A | Low-Risk | 2 | Male | 82 | 2.64 |
| Patient B | Low-Risk | 2 | Male | 73 | 11.08 |

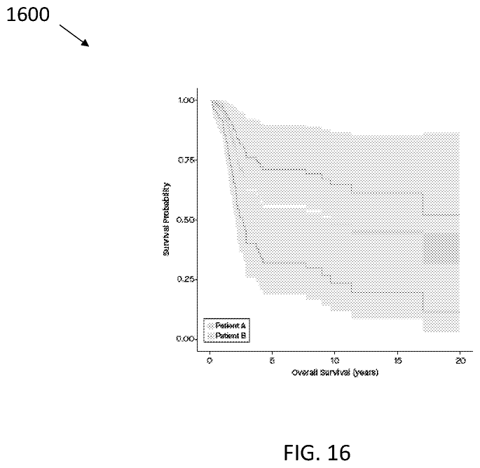

Referring now collectively to Table 4 above and graph 1600 in FIG. 16, personalized survival predictions are presented that were generated by a serum TM-based prognostic classifier. While graph 1600 in FIG. 16 was generated using data generated from blood serum samples, as described above, other suitable liquid biopsy samples, such as plasma or whole blood samples, may be used instead. Graph 1600 in FIG. 16 was produced using multivariable Cox regression. These personalized survival predictions may be able to provide a more detailed and/or complete prediction of survival outcomes over a patient's lifespan as compared to categorical information derived from conventional IPSS-R scores. The data for Table 4 and graph 1600 were generated from two patients, A and B, who were held out from classifier training, and from a multivariate Cox regression model with age, sex, and methylation prognostic score as covariates.

With reference to Table 4, using conventional IPSS-R, Patient A and Patient B were both categorized as low-risk individuals and were both assigned the same IPSS-R score, i.e., 2. However, data generated by the prognostic classifier revealed that Patient A had a much shorter expected survival expectancy (i.e., 2.64 years) than Patient B (i.e., 11.08), despite being assigned the same IPSS-R score. Under the conventional approach, both patients may have been provided with similar survival outcomes and may have been recommended similar treatment options despite the fact that their predicted survival outcomes using the new methodology described herein were vastly different.

With respect to graph 1600, personal survival curves are illustrated that were generated for Patients A and B over each of their lifespans. These curves may provide a visual representation of the predicted survival outcomes for each of these patients that may be more informative than just a conventional IPSS-R score or a categorical classifier. For instance, examination of graph 1600 reveals that at approximately 2.5 years, the survival probability of Patient B is greater than 75%, whereas the survival probability for Patient A is approximately 35%. Stated differently, the survival outlook for Patient A falls dramatically after approximately 2.5 years, whereas the survival outlook for Patient B remains above 50% for at least the next 20 years. The more granular information obtained from graph 1600 may help better inform treatment options for both of these patients (e.g., in terms of time, convenience, comfortability, etc.).

Taken collectively, the novel techniques for liquid biopsy, e.g., blood sample, analysis described herein may be utilized in conjunction with bone marrow testing and/or may replace the necessity for bone marrow testing. Additionally, although the classifiers described herein generally produced results that classified a patient's survival outcome into two categories based upon a binary 3-year threshold (e.g., short survival outcome under 3 years or long survival outcome over 3 years), such a classification methodology is not limiting. More particularly, the classifiers may be trained to generate scores that predict patient survival by classifying the patient into more than two categories. Further, although a threshold of 3 years was selected in the examples above, any suitable survivor length threshold or thresholds may be selected to distinguish between the two or more categories. And, in some aspects, the classifiers may be trained to generate scores that predict patient survival by classifying the patient into and/or by providing survival probabilities over the remaining course of a patient's life, as was done in reference to graph 1600 in FIG. 16.

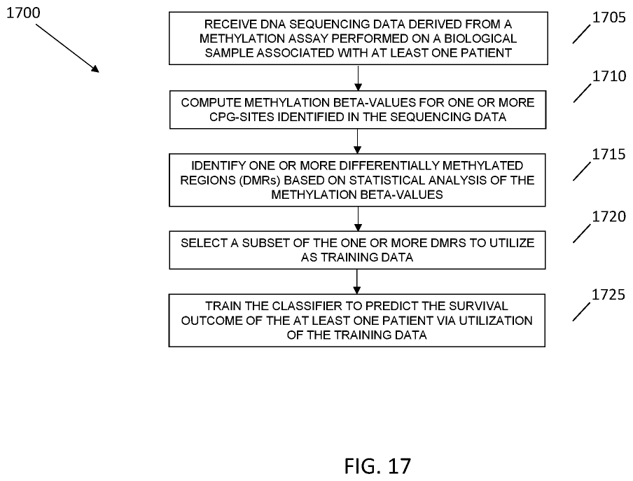

Referring now to FIG. 17, an exemplary process flow 1700 is depicted for building a classifier to predict a survival outcome in at least one patient diagnosed with MDS, according to one or more aspects of the present disclosure. The exemplary process flow 1700 may be implemented, e.g., by some or all components of system environment 100.

At step 1705, DNA sequencing data derived from a methylation assay performed on a biological sample associated with a patient may be received at the system environment 100. In an aspect, the methylation assay performed on the biological sample may be either a cell-free DNA TM assay or a bone marrow WGBS assay. In the case of the former, the biological sample may be a blood plasma or serum sample. In the case of the latter, the biological sample may be a bone marrow tissue sample. It is important to note that the foregoing sample types should not be considered limiting and other sample types may be utilized, e.g., a blood sample (e.g., a serum sample, a plasma sample, a whole blood sample), a urine sample, a saliva sample, a tissue sample, a bone marrow sample, etc.

At step 1710, methylation beta values may be computed for some or all of the CpG sites encompassed by the sequencing data. The methylation beta values are continuous variables between 0 and 1 that represent the percentage of methylation associated with a given CpG site. These beta values may be computed utilizing techniques and formulas previously described herein.

At step 1715, the computed beta values may be used to identify one or more DMRs. These DMRs represent genomic regions that exhibit statistically significant differences in methylation patterns between different groups or conditions. For example, DMRs in a patient afflicted with MDS may exhibit different methylation states than those same DMRs in patients not afflicted with MDS.

At step 1720, certain DMRs and/or individual CpG sites may be selected as training features in a feature selection process. In an aspect, the feature selection process may reduce the dimensionality of a dataset by eliminating irrelevant or redundant features. In the context of the methylation approaches described herein, feature selection may involve the identification of a subset of DMRs, alongside individual CpG site beta values, that may be more relevant to the prediction of an individual's survival status. By incorporating both DMRs and beta values as features in the training and prediction process, the model may leverage the information from different scales of methylation data. DMRs capture larger-scale methylation patterns associated with specific genomic regions, whereas beta values provide detailed information about methylation levels at individual CpG sites. In an aspect, one approach that may be leveraged to perform feature selection may be principal component analysis.

At step 1725, the selected features may be utilized to train a machine learning classifier to predict a survival outcome of a patient. More particularly, the classifier may be trained to recognize patterns and correlations between DMRs in bone marrow tissue and/or serum and corresponding survival outcomes in patients. In some aspects, the classifier may be configured to generate a score that represents a binary indication of short survival (e.g., under 3 years) or long survival (greater than 3 years) for individual's diagnosed with MSD. In an aspect, these risk scores may further be used as inputs downstream to inform treatment plans. In other aspects, the score output by the classifier may be associated with a particular category of survival length and/or may be utilized to calculate the probabilities of survival over a predetermined period of time.

### Correcting for Hypomethylating Agent Treatment Effects

In an aspect, the effects of hypomethylating agents (HMAs) may potentially affect the ability of the prognostic classifier described herein to predict patient survival outcomes. More particularly, HMAs are cytosine nucleoside analogs that inhibit DNMT by incorporating into nucleic acids during the S-phase of DNA replication, then forming irreversible covalent bonds with DNMT leading to DNMT trapping, and ultimately blocking its function and causing its depletion. These HMAs may be administered to some patients that have MDS, and their mechanism of action (i.e., the inhibition of DNMT) may lead to changes in a patient's methylation profile. Exemplary classes of drugs utilized for MDS treatment include Vidaza (Azacitidine) and Dacogen (Decitabine). The former is a ribonucleoside that is only incorporated into 10% of DNA, whereas the other 90% of the drug is incorporated into RNA. The latter is a deoxyribonucleoside that is incorporated into DNA and is a more potent hypomethylating agent than Vidaza at equivalent concentration.

TABLE 5

| | Number of Samples | Percent of Total |
|---|---|---|
| Current HMA | 25 | 19.6% |
| Post HMA | 16 | 12.7% |
| Untreated | 86 | 67.7% |

TABLE 6

| | Number of Samples | Percent of Total |
|---|---|---|
| HMA+ | 41 | 32.3% |
| HMA− | 86 | 67.7% |

Tables 5 and 6 above collectively provide statistical information about the patients treated by an HMA agent. More particularly, Table 5 provides a breakdown of the patients that are currently undergoing HMA treatment, the patients that have previously undergone HMA treatment, and the patients that have not undergone HMA treatment of any kind by the time of sample collection. Referring to Table 6, the patients that are either currently undergoing HMA treatment or have previously undergone HMA treatment are grouped together as HMA+, while the patients that have never received HMA treatment are placed in the HMA− group. As can be observed from Table 5, nearly one-third of the patients involved in the study are currently undergoing, or have previously received, HMA treatment.

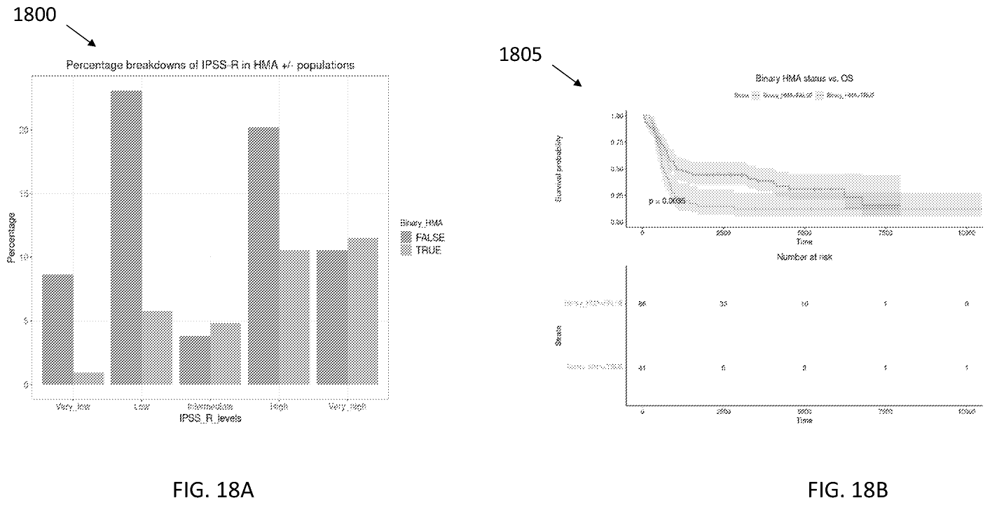

Referring now to FIG. 18A, graph 1800 is provided that illustrates a relationship between the percentage of HMA−/HMA+ samples in each IPSS-R level. For instance, patients receiving very low risk and low risk IPSS-R scores were generally associated with the HMA− grouping (i.e., those individuals that had never received an HMA treatment) because, as expected, there would be little reason to receive for them to seek out or be recommended an HMA treatment due to their low chance of developing MDS. Conversely, a significantly greater percentage of patients receiving intermediate, high, and very high risk scores were associated with the HMA+ grouping (i.e., those individuals that had previously received or are currently receiving HMA treatment).

Referring now to FIG. 18B, graph 1805 illustrates the survival rates for patients associated with both the HMA− and HMA+ groupings. As can be generally observed, patients that have had or are currently undergoing HMA treatment, indicating that they likely had intermediate to very high risk of developing MDS, died earlier than the patients who have never had HMA treatment. For instance, after 2500 days (approximately 6.85 years), approximately 38% of the patients associated with the HMA− grouping were alive, whereas only approximately 15% of the patients associated with HMA+ grouping were alive.

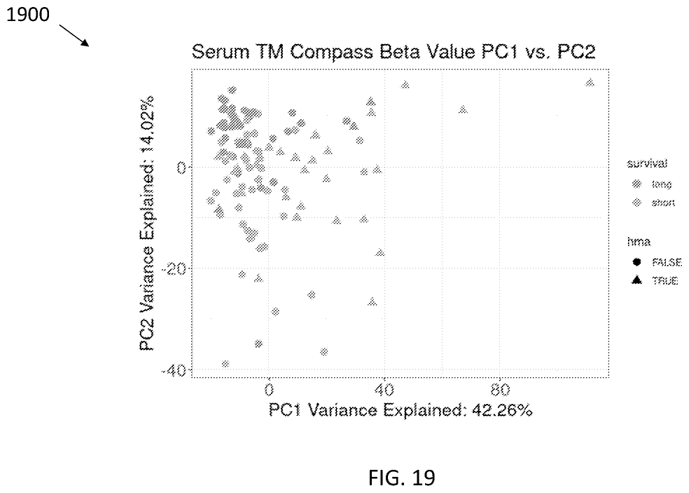

In an aspect, although it was determined that the HMA status of a patient is not associated with survival after accounting for IPSS-R levels, an HMA treatment may still affect the methylation features in a sample to some degree. For instance, referring now to FIG. 19, PCA plot 1900 is presented of the methylation beta values in the blood serum TM dataset. The triangles represent patients who have previously had or were currently undergoing HMA treatment at the time of sample collection, and the circles represent those patients who have not had an HMA treatment at the time of sample collection. As can be observed, the majority of the outliers in plot 1900 correspond to those patients in the short survivor group who have been associated with an HMA treatment, e.g., the teal triangle data points.

In an aspect, the effects of HMA treatment may be corrected by regressing out the effects of HMA from whichever feature matrix is being utilized (e.g., beta-value matrix) utilizing a linear correction. This correction may be performed in order to assess what the survival classifier performance would be without the influence of HMA. To perform linear correction, the methylation beta values may first be converted to logit-transformed beta values, or “M-values”. More particularly, because beta values are bounded between 0 and 1, performance of statistical regression processes using untransformed beta values may be problematic in the context of DNA methylation analysis. Specifically, the variance of the beta values is usually smaller near the boundaries than near the middle of the interval (0, 1), implying that the homoscedasticity assumption in Gaussian regression is violated. The conversion of beta values to M-values may be facilitated by employing incorporated available values into known formulas. For instance, the methylation beta value may be calculated as beta=M/(M+U+a), where M is methylated intensity, U is unmethylated intensity, and “a” is a constant offset (by default, a=100). The M value is calculated as M=log₂((M+a)/(U+a)). The values M or U are usually greater than 1000, so “a” may be negligible for most probes. Accordingly, if a=0, then the M-value may be calculated with: M=log₂(beta)/(1-beta). Once the methylation beta values at each CpG site are converted into M-values, a per-feature linear regression may be performed, and the calculated residuals may then subsequently be utilized as input to downstream analysis tasks, such as PCA analysis and classifier training.

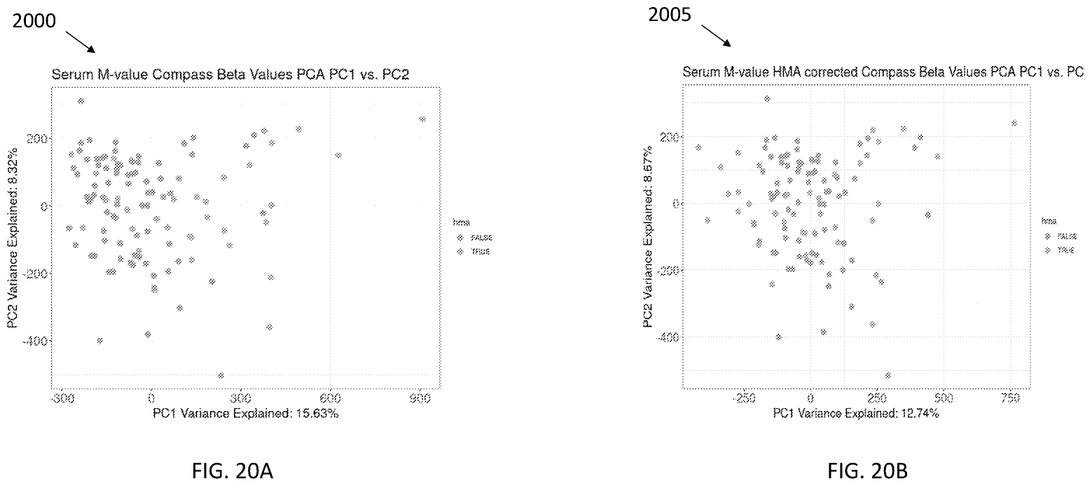

Referring now to FIGS. 20A and 20B, graphs 2000 and 2005, respectively, illustrate the effects of HMA correction in blood serum TM samples. More particularly, graph 2000 represents the uncorrected data and illustrates that clustering exists between the HMA-affected samples and the non-HMA-affected samples. Conversely, graph 2005 represents the corrected data and illustrates a more spread out dataset in which the clustering by HMA has been removed, thereby indicating that the correction process described herein has worked.

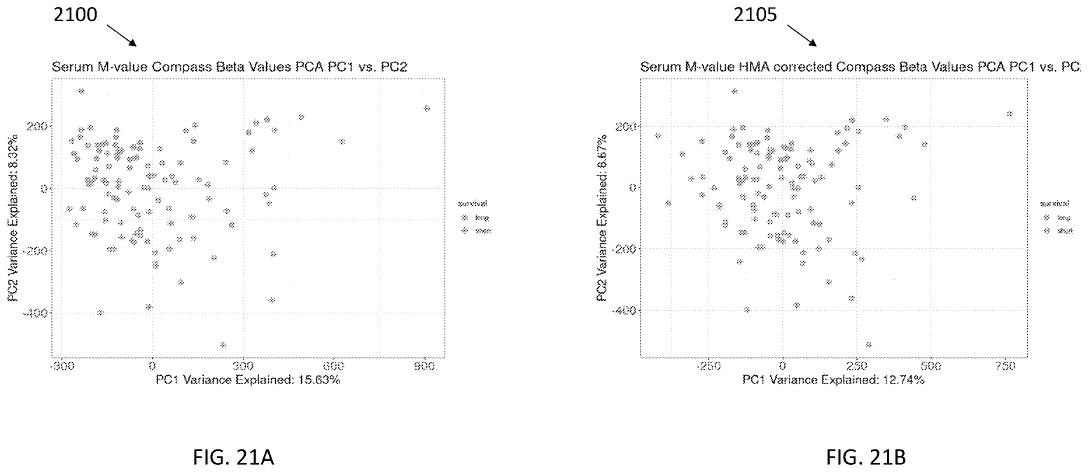

Referring now to FIGS. 21A and 21B, graphs 2100 and 2105, respectively, show that stratification by survival still remains after regressing out the HMA effect in serum TM samples. More particularly, graph 2000 represents the uncorrected data and illustrates that clustering generally exists between short- and long-term survivors, e.g., because short-term survivors were more akin to receiving HMA treatments. Conversely, graph 2105 represents the corrected data and illustrates that clustering between short- and long-term survival samples are reduced.

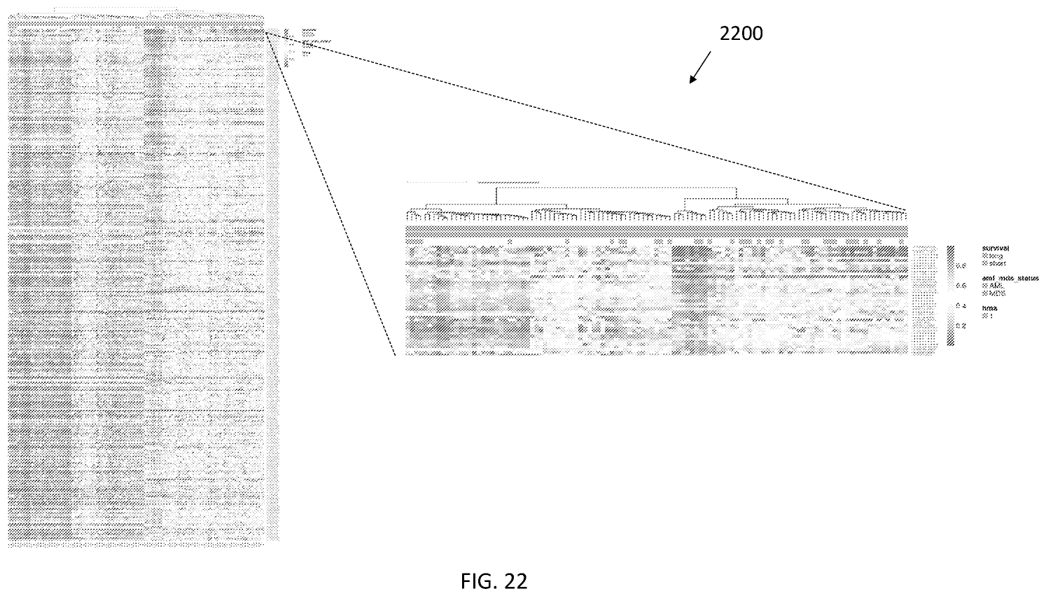

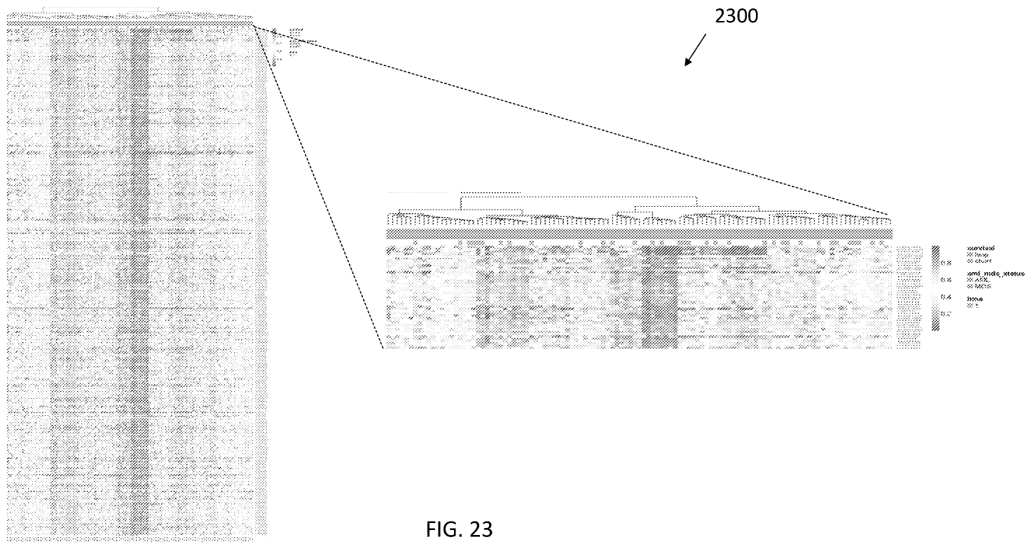

Referring now to FIGS. 22 and 23, heatmaps 2200 and 2300, respectively, show the actual effect of the HMA correction for blood serum TM samples. More particularly, heatmap 2200 corresponds to the graphical representation of the methylation states at each differentially methylated CpG site in the uncorrected data. As can be observed, without the HMA correction, clustering in columns is present, e.g., HMA treated datasets are clustered to the right. Conversely, heatmap 2300 corresponds to the graphical representation of the methylation states at each differentially methylated CpG site in the corrected data. As can be observed, with the HMA correction, the HMA+ samples are more distributed.

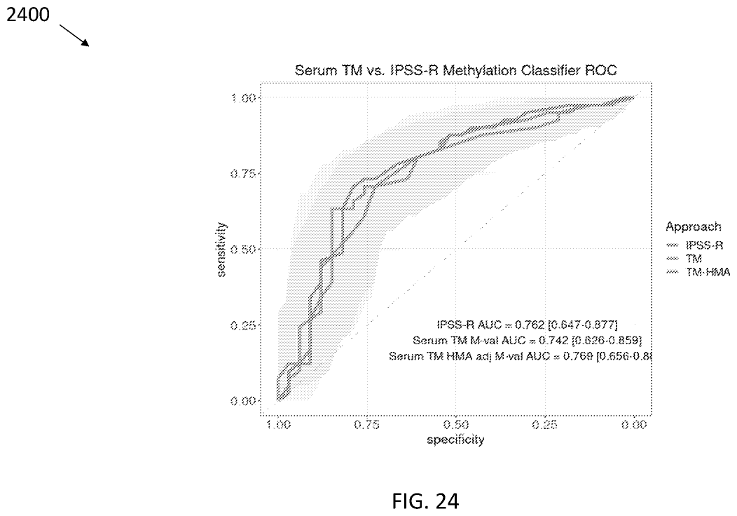

Referring now to FIG. 24, graph 2400 illustrates that HMA-corrected M-values show similar performance as unadjusted M-values for blood serum TM samples. More particularly, utilizing the nested cross-validation PC Random Forest prediction approach to assess performance, it can be observed that although the overall performance of the classifier dropped slightly when utilizing M-values instead of beta values, the classifier still performed well in comparison to IPSS-R. Therefore, it is indicated that the utilization of M-values and HMA-corrected M-values does not result in a large loss of predictive ability.

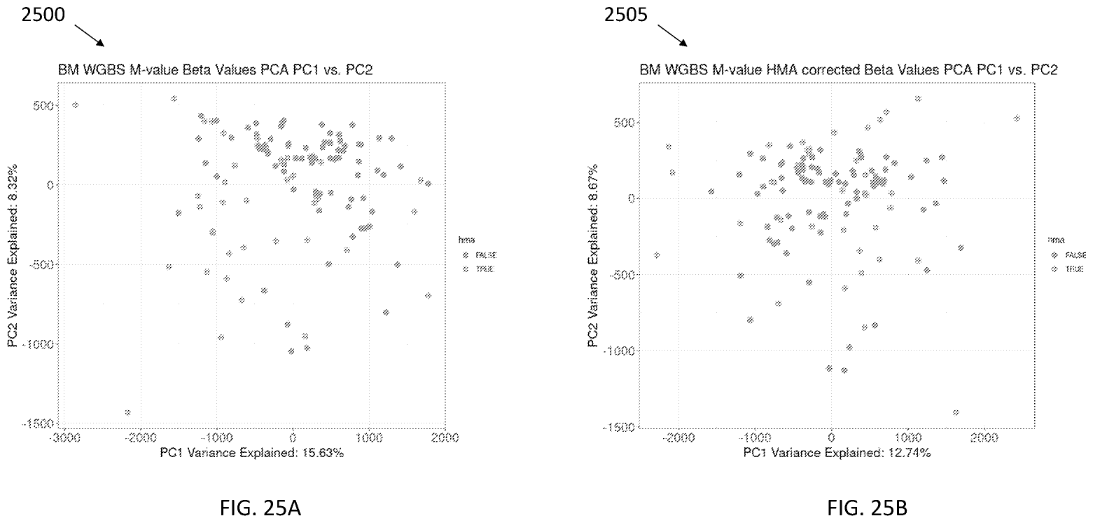

Referring now to FIGS. 25A and 25B, graphs 2500 and 2505, respectively, illustrate the effects of HMA correction in bone marrow WGBS samples. In general, linear regression to remove HMA effects on bone marrow WGBS M-values appears effective. More particularly, graph 2500 represents the uncorrected data and illustrates that clustering exists between the HMA-affected samples and the non-HMA− affected samples. Conversely, graph 2505 represents the corrected data and illustrates a more spread out dataset in which the clustering by HMA has been removed, thereby indicating that the correction process described herein has worked.

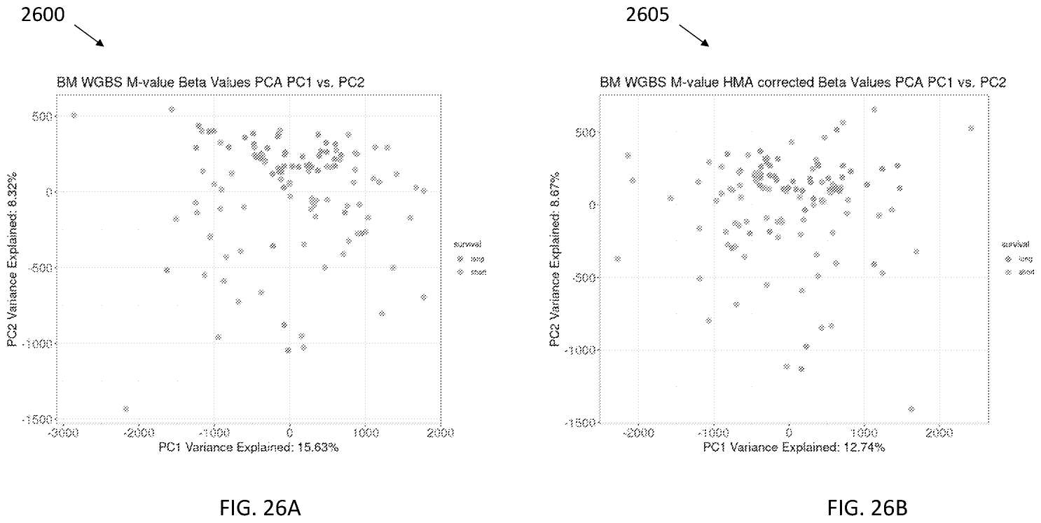

Referring now to FIGS. 26A and 26B, graphs 2600 and 2605, respectively, show that stratification by survival still remains after regressing out the HMA effect in bone marrow WGBS samples. More particularly, graph 2600 represents the uncorrected data and indicates that clustering generally exists between short- and long-term survivors, e.g., because short-term survivors were more likely to receiving HMA treatments. Conversely, graph 2605 represents the corrected data and indicates that clustering between short- and long-term survival samples are reduced.

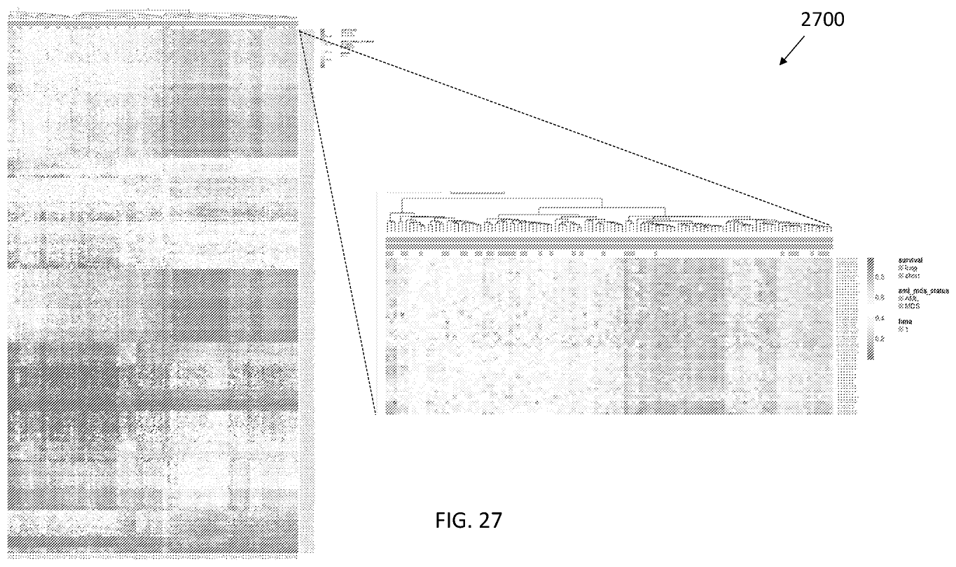

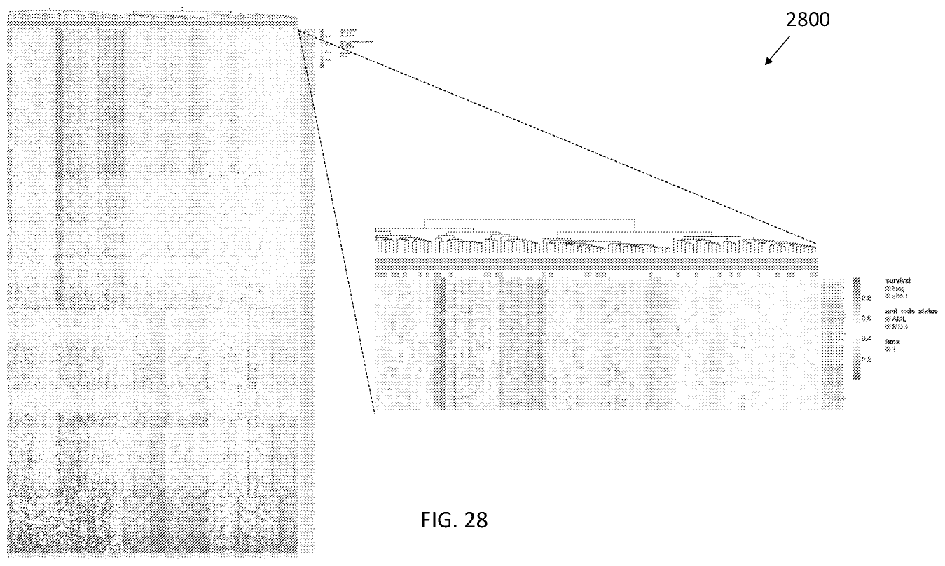

Referring now to FIGS. 27 and 28, heatmaps 2700 and 2800, respectively, show the actual effect of the HMA correction. More particularly, heatmap 2700 corresponds to the graphical representation of the methylation states at each differentially methylated CpG site in the uncorrected data. As can be observed, without the HMA correction, clustering in columns may be present, e.g., HMA treated datasets are clustered to the left. Conversely, heatmap 2800 corresponds to the graphical representation of the methylation states at each differentially methylated CpG site in the corrected data. As can be observed, with the HMA correction, the HMA+ samples are more distributed. Additionally, it can further be observed from graphs 2700 and 2800 that the correction methodology for bone marrow WGBS has shifted the beta values such that certain methylation patterns in the pre-corrected data are no longer retained post correction.

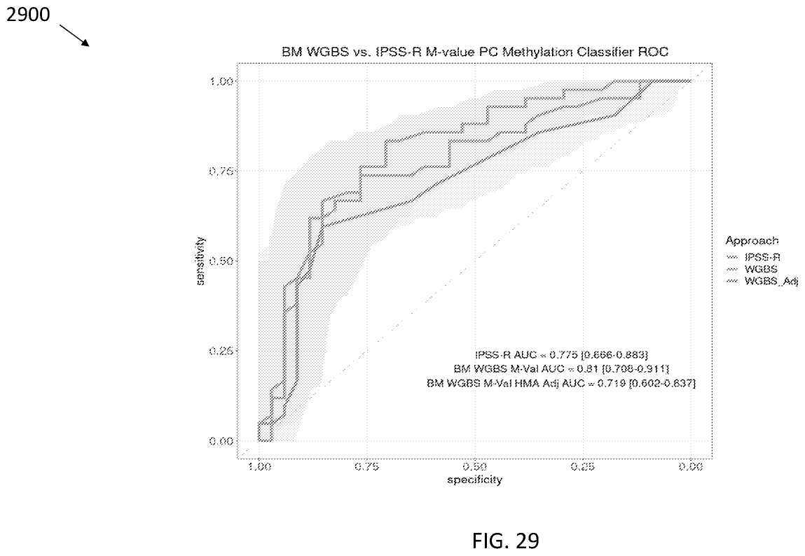

Referring now to FIG. 29, graph 2900 illustrates that HMA-corrected M-values may degrade bone marrow WGBS classifier performance. Such a degradation may result, for instance, because the HMA targets myeloid cells that are mostly concentrated in the bone marrow, which may correspondingly have a bigger effect on the methylation states of bone marrow based CpG sites.

In general, any process discussed in this disclosure that is understood to be computer-implementable may be performed by one or more processors of a computer system, such as system environment 100, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 100, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

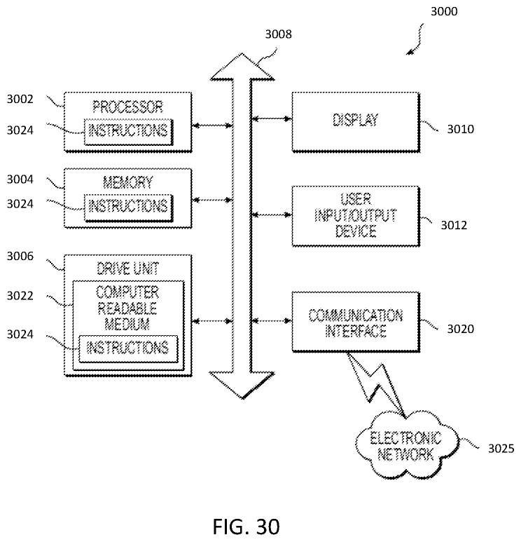

FIG. 30 is a simplified functional block diagram of a computer system 3000 that may be configured as a computing device for executing the processes described herein, according to exemplary embodiments of the present disclosure. FIG. 30 is a simplified functional block diagram of a computer that may be configured as according to exemplary embodiments of the present disclosure. In various embodiments, any of the systems herein may be an assembly of hardware including, for example, a data communication interface 3020 for packet data communication. The platform also may include a central processing unit (“CPU”) 3002, in the form of one or more processors, for executing program instructions. The platform may include an internal communication bus 3008, and a storage unit 3006 (such as ROM, HDD, SDD, etc.) that may store data on a computer readable medium 3022, although the system 3000 may receive programming and data via electronic network 3025 (e.g., voice, video, audio, images, or any other data over the electronic network 3025). The system 3000 may also have a memory 3004 (such as RAM) storing instructions 3024 for executing techniques presented herein, although the instructions 3024 may be stored temporarily or permanently within other modules of system 3000 (e.g., processor 3002 and/or computer readable medium 3022). The system 3000 also may include input and output ports 3012 and/or a display 3010 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for building a classifier to predict a survival outcome in at least one patient diagnosed with Myelodysplastic Syndrome (MDS), comprising:
receiving, at a computing device, DNA sequencing data derived from a methylation assay performed on a biological sample associated with the at least one patient;
computing, using a processor associated with the computing device, methylation beta values for one or more CpG-sites identified in the sequencing data;
identifying, using the processor, one or more differentially methylated regions (DMRs) based on statistical analysis of the methylation beta-values for the one or more CpG-sites;
selecting, using the processor and via a feature selection process, a subset of the one or more DMRs to utilize as training data; and
training, using the processor and the training data, the classifier to predict the survival outcome of the at least one patient.

2. The method of claim 1, wherein the methylation assay is a cell-free DNA targeted methylation assay and the biological sample is one of: a blood plasma sample or a blood serum sample.

3. The method of claim 1, wherein the methylation assay is a whole-genome bisulfite sequencing (WGBS) assay and wherein the biological sample is bone marrow tissue.

4. The method of claim 1, wherein the feature selection process corresponds to a principal component analysis technique.

5. The method of claim 1, wherein the classifier is a principal component random forest classifier.

6. The method of claim 1, further comprising assessing a performance of the classifier utilizing nested cross-validation.

7. The method of claim 6, wherein the nested cross-validation is further utilized to optimize hyperparameters in the training data.

8. The method of claim 1, wherein the training the classifier comprises configuring the classifier to generate a score that is associated with the survival outcome.

9. The method of claim 1, wherein the survival outcome is a binarized survival outcome designation.

10. The method of claim 1, wherein the training data further includes one or more clinical variables associated with the at least one patient.

11. A system for building a classifier to predict a survival outcome in at least one patient diagnosed with Myelodysplastic Syndrome (MDS), the system comprising:
one or more processors;
one or more computer readable media storing instructions that are executable by the one or more processors to perform operations to:
receive, at a computing device associated with the system, DNA sequencing data derived from a methylation assay performed on a biological sample associated with the at least one patient;
compute, using the one or more processors, methylation beta values for one or more CpG-sites identified in the sequencing data;
identify, using the one or more processors, one or more differentially methylated regions (DMRs) based on statistical analysis of the methylation beta-values for the one or more CpG-sites;
select, using the one or more processors and via a feature selection process, a subset of the one or more DMRs to utilize as training data; and
train, using the one or more processors and the training data, the classifier to predict the survival outcome of the at least one patient.

12. The system of claim 11, wherein the methylation assay is a cell-free DNA targeted methylation assay and the biological sample is one of: a blood plasma sample or a blood serum sample.

13. The system of claim 11, wherein the methylation assay is a whole-genome bisulfite sequencing (WGBS) assay and wherein the biological sample is bone marrow tissue.

14. The system of claim 11, wherein the feature selection process corresponds to a principal component analysis technique.

15. The system of claim 11, wherein the classifier is a principal component random forest classifier.

16. The system of claim 11, wherein the operations further comprise instructions to:
assess a performance of the classifier utilizing nested cross-validation.

17. The system of claim 16, wherein the nested cross-validation is further utilized to optimize hyperparameters in the training data.

18. The system of claim 11, wherein the operations to train the classifier further comprise operations to:
configure the classifier to generate a score that is associated with the survival outcome.

19. The system of claim 11, wherein the survival outcome is a binarized survival outcome designation.

20. A non-transitory computer-readable medium storing computer-executable instructions which, when executed by a system, cause the system to perform operations comprising:
receiving, at a computing device associated with the system, DNA sequencing data derived from a methylation assay performed on a biological sample associated with at least one patient;
computing, using a processor associated with the computing device, methylation beta values for one or more CpG-sites identified in the sequencing data;
identifying, using the processor, one or more differentially methylated regions (DMRs) based on statistical analysis of the methylation beta-values for the one or more CpG-sites;
selecting, using the processor and via a feature selection process, a subset of the one or more DMRs to utilize as training data; and
training, using the processor and the training data, a classifier to predict a survival outcome of the at least one patient.
